(19) United States
(12) Patent Application Publication
(10) Pub. No.: US 2009/0306769 A1
(43) Pub. Date: Dec. 10, 2009

(54) MEDICAL BALLOON MADE WITH HYBRID POLYMER-CERAMIC MATERIAL AND METHOD OF MAKING AND USING THE SAME

(75) Inventors: Scott Schewe, Eden Prairie, MN (US); Michele Zoromski, Minneapolis, MN (US)

Correspondence Address: VIDAS, ARRETT & STEINKRAUS, P.A., SUITE 400, 6640 SHADY OAK ROAD, EDEN PRAIRIE, MN, 55344, US

(73) Assignee: BOSTON SCIENTIFIC SCIMED, INC., Maple Grove, MN (US)

(21) Appl. No.: 12/134,357

(22) Filed: Jun. 6, 2008

(51) Int. Cl. A61F 2/82; A61M 25/10; A61M 25/098; A61M 25/00

(52) U.S. Cl. 623/1.34; 604/96.01; 604/523; 623/1.42; 604/103.1; 427/2.3

(57) ABSTRACT

A medical device such as a catheter, stent or balloon, or a component thereof is formed by depositing an inorganic-organic hybrid composite material on an eliminatable shape form and removing the shape form, leaving the medical device or component thereof formed of the inorganic-organic hybrid composite material. Multiple layers of the inorganic-organic hybrid composite material can be used in the formation. A particular structure is an expandable balloon member for a catheter assembly.

### FIELD OF THE INVENTION

The present invention relates generally to the field of insertable or implantable medical devices, in particular, expandable medical devices, and to methods of making the same.

### BACKGROUND OF THE INVENTION

Medical devices formed from polymer compositions are manufactured using conventional polymer thermoforming techniques including extrusion, injection molding, stretch blow molding, and the like.

High tensile strengths are important in medical device balloons because the balloons are thin walled structures that for some applications, such as percutaneous transluminal coronary angioplasty (PTCA), the balloons may be inflated at high pressures. Keeping wall thickness of the balloons to a minimum is advantageous for advancing the balloon through the vasculature of the patient. Similar factors are important in catheter shaft materials.

For some thermoforming techniques, such as blow molding, some portions of the balloon, such as the cone portions, have a thicker wall than the central section resulting in a balloon that has a larger profile during folding.

Thus, there are a variety of complex factors taken into account during balloon fabrication.

U.S. Patent Publication No. 2005/0015046 discloses processes for forming articles, particularly medical devices, from radiation curable compositions in which a pattern-wise curing is used to form the device or coatings thereon.

### SUMMARY OF THE INVENTION

In one aspect, the present invention relates to medical devices, particularly expandable balloon members or other catheter components such as catheter shafts, tips, and so forth, formed from a composite material that includes at least one polymer and at least one sol-gel derived ceramic.

In another aspect, the present invention relates to a method of forming medical devices, particularly expandable balloon members or other catheter components including providing an eliminatable shape form, disposing at least one first layer over the eliminatable shape form, the first layer defining the shape of the medical device, the first layer formed from a composite material that includes at least one polymer and at least one sol-gel derived ceramic, and eliminating the shape form. Multiple layers of the composite material can be disposed onto the first layer in the same fashion prior to eliminating the shape form.

These and other aspects, embodiments and advantages of the present invention will become immediately apparent to those of ordinary skill in the art upon review of the Detailed Description and Claims to follow.

### BRIEF DESCRIPTION OF THE DRAWING

FIG. 1 is a longitudinal cross-section of a catheter assembly having an expandable balloon member mounted on the distal end thereof.

FIG. 2 is longitudinal cross-section of an expandable balloon member formed according to the invention.

FIG. 3 is a side view of an eliminatable shape form that may be used to form an expandable balloon member in accordance with the invention.

FIG. 4 is a longitudinal cross-sectional view of an eliminatable shape form having a first layer including a composite material according to the invention disposed thereon.

FIG. 5 is a longitudinal cross-sectional view of a shape form having first and second layers of composite materials according to the invention disposed thereon.

### DETAILED DESCRIPTION OF THE INVENTION

While this invention may be embodied in many different forms, there are described in detail herein specific preferred embodiments of the invention. This description is an exemplification of the principles of the invention and is not intended to limit the invention to the particular embodiments illustrated.

All US patents and applications and all other published documents mentioned anywhere in this application are incorporated herein by reference in their entirety. Any copending patent applications, mentioned anywhere in this application are also hereby expressly incorporated herein by reference in their entirety.

In one aspect, the present invention provides for insertable and/or implantable medical devices formed from a composite material including at least one polymer and at least one sol-gel derived ceramic.

Such insertable and/or implantable medical devices are employed for diagnosis, for systemic treatment, or for the localized treatment of any tissue or organ. The devices may be employed for treatment in a variety of body systems including, but not limited to, coronary and peripheral vascular system (referred to overall as “the vasculature”), renal system including the kidneys, the urogenital system, including, urethra, ureters, prostate, vagina, uterus and ovaries, esophageal system including trachea, esophagus and larynx, neurological system including the brain, etc.

In some embodiments, the present invention provides for catheter assemblies wherein at least a portion thereof or the entire device is formed from a composite material including at least one polymer and at least one sol-gel derived ceramic.

In one specific embodiment, expandable balloon members, disposed about the distal end of catheter assemblies, are formed from a composite material including at least one polymer and at least one sol-gel derived ceramic.

The composite material is advantageous in that it can provide for enhanced mechanical characteristics including enhanced strength, toughness and/or abrasion resistance.

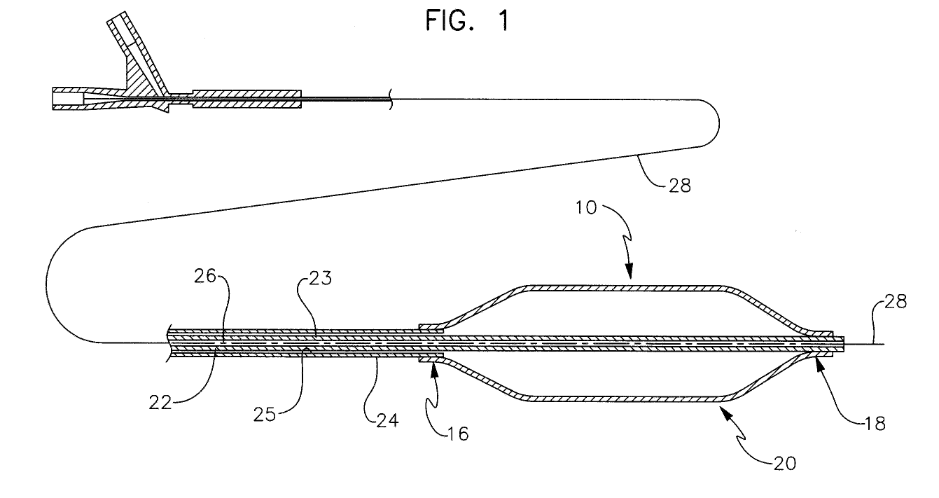

Turning now to the drawings, FIG. 1 illustrates an expandable balloon member 10 mounted at the distal end of a catheter assembly 20. In this embodiment, balloon 10 is shown formed with a composite first layer 12, including at least one polymer and at least one sol-gel derived ceramic. Catheter 20 is a representative rapid exchange or single-operator-exchange (SOE) angioplasty balloon.

In this embodiment, catheter 20 has an elongate dual shaft assembly 18 including an inner shaft 22 and an outer shaft 24. The outer shaft 24 is coaxially disposed about inner shaft 22 and defines an annular inflation lumen 25. The inner lumen 26 of inner shaft 22 defines guide wire lumen through which guide wire 28 is disposed. Catheter assembly 20 also includes a manifold assembly 30 connected to proximal end of shaft assembly 18. Manifold assembly 30, is further shown with a strain relief 32. This is only an illustration of such a catheter assembly and is not intended to limit the scope of the present invention. Numerous structures are known to those of skill in the art, any of which may be employed herein. Balloon catheters are discussed, for example, in commonly assigned U.S. Pat. Nos. 6,113,579, 6,517,515, 6,514,228, each of which is incorporated by reference herein in its entirety.

Any of the catheter components may be formed of the composite material including at least one polymer and at least one sol-gel derived ceramic according to the invention. These materials are explained in detail below. For example, inner shaft 22 and outer shaft 24, or portions thereof, as well as a catheter tip (not shown) may be constructed of the composite material. In this embodiment, expandable balloon member 10 is constructed of at least one layer of the composite material according to the invention. Expandable balloon member 10 may be constructed of a plurality of layers of the composite material as well, and in some embodiments, it may be preferable to do so to provide a desired balance of properties such as flexibility and strength.

FIG. 2 is a longitudinal cross-section of an expandable balloon member 10 formed of a first composite layer 12 including at least one polymer and at least one sol-gel derived ceramic. Multiple layers of the composite material including second, third and fourth layers, etc. may be deposited over first layer 12.

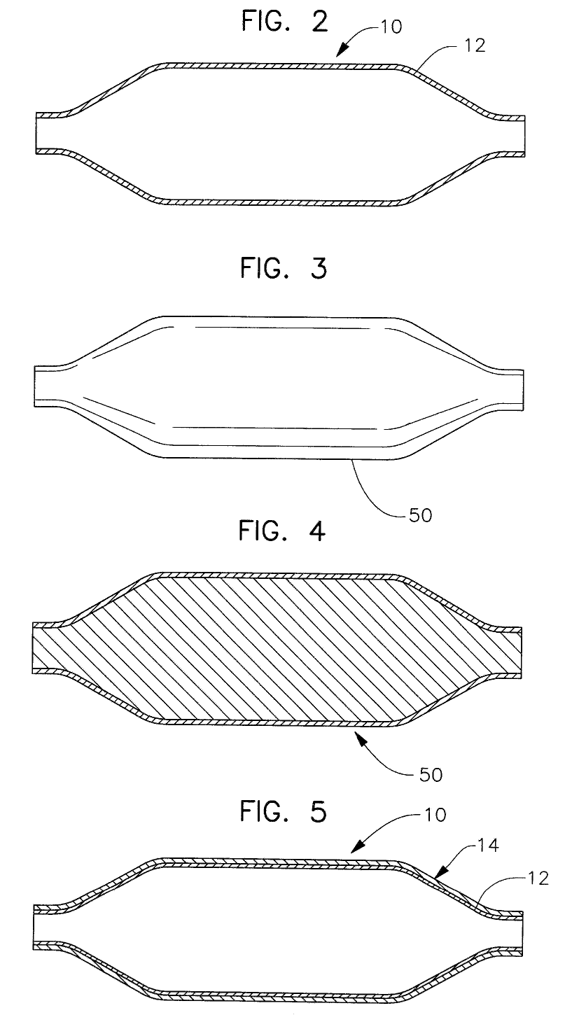

Balloon 10 may be formed using a shape form 50 shown in FIG. 3 which defines the shape of balloon 10, but which is eliminated after use. A first layer 12 may be provided over the eliminatable shape form, the first layer defining shape of balloon 10. A second layer 14 is shown provided over the shape form in FIG. 5. Three, four and more layers may be provided. This can be achieved in a step wise manner by multiple dippings, sprayings, paintings, and so forth.

Of course, the multiple layers do not have to be the same hybrid composition. For example, if desired, an organic polymer composition could be applied to certain portions of the balloon layer, for example, at the waist, to facilitate melting/recrystallization during laser or heat welding processes, for example, in securing the balloon to catheter shaft(s).

The shape form 50 may be formed from ice, wax, soluble polymer, or other dissolvable materials. Alternatively, the shape form may also be deflatable, and upon application of negative pressure, deflated and removed from the now formed balloon member.

The medical device may be formed by a method that includes the steps of providing an eliminatable shape form, forming a composition that includes a hydrolyzed and partially condensed sol-gel ceramic precursor, applying the composition to the shape form, condensing the hydrolyzed, partially condensed ceramic precursor to form a composite inorganic-organic sol-gel material, and eliminating the shape form. Suitable methods of forming the composite material are discussed in detail below.

Most of these methods involve hydrolysis and condensation reactions which lead to the formation of a suspension containing a ceramic phase, which is analogous to the “sol” that is formed in sol-gel processing. For many of these methods, this suspension will also include a polymer phase. The sol can then be deposited onto the eliminatable shape form. Subsequent removal of water (as well as any other solvent species that may be present), results in the formation of a solid phase, which is analogous to the “gel” in sol-gel processing.

In techniques where a polymer is present which has thermoplastic characteristics, the composite material may be heated to form a melt for further processing.

Useful techniques for applying sols or melts onto the eliminatable shape form may include spray coating, coating with an applicator (e.g., by roller or brush), spin-coating, dip-coating, ink-jet printing, screen printing, extrusion, etc. whereby a “wet gel” is formed.

Sol-gel processes are suitable for use in conjunction with polymers and their precursors (as well as therapeutic agents, in some embodiments of the invention), for example, because they can be performed at ambient temperatures. A detailed review of various techniques for generating polymeric-ceramic composites can be found, for example, in G. Kickelbick, “Concepts for the incorporation of inorganic building blocks into organic polymers on a nanoscale” Prog. Polym. Sci., 28 (2003) 83-114. Other published documents describing polymer-ceramic nanocomposite materials include: P. Xu, “Polymer-Ceramic Nanocomposites,” Encyclopedia of Materials: Science and Technology, Elsevier Science Ltd. (2000); L. Shen, et al, “In situ polymerization and characterization of polyamide-6/silica nanocomposite derived from water glass,” Polymer International, 53, 1153-1160 (2004); K. Haas et al, “Hybrid Organic/Organic Polymers with Nanoscale Building Blocks; Precursors, Processing, Properties and Applications,” Rev. Adv. Mater. Sci., 5 (2003) 47-52; R. Zoppi et al, “Hybrids of Poly(ethylene oxide-b-amide-6) and ZrO.sub.2 Sol-gel: Preparation, Characterization, and Application in Processes of Membranes Separation,” Advances in Polymer Technology, Vol. 21, No. 1, 2-16 (2002); H. Huang et al, “Structure-property behaviour of hybrid materials incorporating tetraethoxysilane with multifunctional poly(tetramethylene oxide)” Polymer, 30, 2001-2012 (1989); and J. Pyun et al, “Synthesis of Nanocomposite Organic/Inorganic Hybrid Materials Using Controlled/‘Living Radical’ Polymerization,” Chem. Mater., 13:3436-3448 (2001), all of which are also expressly incorporated herein by reference in their entirety.

It is known, for example, to generate polymeric-ceramic composites by conducting sol-gel processing in the presence of a preformed polymer, which techniques can be successful, for example, where the polymer is soluble in the sol-forming solution (e.g., a solution containing alkoxy species, such as one containing tetraethoxysilane (TEOS), also known as tetraethylorthosilicate, or tetramethoxysilane (TMOS), also known as tetramethylorthosilicate, and/or where the polymer has substantial non-covalent interactions with the ceramic phase (e.g., due to hydrogen bonding between hydroxyl groups and electronegative atoms within the polymeric and ceramic phases), which prevent macroscopic phase separation.

Conversely, it is also known, for example, to impregnate a gel such as a xerogel with monomer and polymerize the monomer within the gel. Analogous to the above, best results are obtained where there are non-covalent interactions between the monomer/polymer and the gel, which are sufficiently strong to prevent macroscopic phase separation.

The composite material can contain bi-continuous polymeric and ceramic phases, domains of a ceramic phase may be dispersed in a polymer matrix, domains of a polymer phase may be dispersed in domains of a ceramic matrix. In some embodiments the best material properties are obtained when the polymer and ceramic are present in bi-continuous phases, that is, where the ceramic and polymer networks interpenetrate, apparently to the molecular level, so that separate domains are not observed under field emission microscopy or even under transmission electron microscopy. When a separate dispersed phase is present, it desirably will be of nanoscale dimension by which is meant that at least one cross-sectional dimension of the dispersed phase (e.g., the diameter for a spherical or cylindrical phase, the thickness for a ribbon- or plate-shaped phase, etc.) is less than 1 micron (1000 nm), for instance in the range of 0.1 nm to 500 nm, or 1-10 nm. A decrease in such dimensions generally results in an increase in the interfacial area that exists between the polymeric and ceramic phases.

In some cases multiple polymer and/or ceramic phases may be present. For example, multiple polymer phases may exist where the composite material includes a block copolymer or a blend of different polymers.

In several particularly beneficial embodiments of the invention, nanoscale phase domains, or bi-continuous phases, are best achieved by providing covalent interactions between the polymeric and ceramic networks. This result can be achieved via a number of known techniques, including the following: (a) providing species with both polymer and ceramic precursor groups and thereafter conducting polymerization and hydrolysis/condensation simultaneously, (b) providing a ceramic sol with polymer precursor groups (e.g., groups that are capable of participation in a polymerization reaction, such as vinyl groups or cyclic ether groups) and thereafter conducting an organic polymerization step, and/or (c) providing polymers with ceramic precursor groups (e.g., groups that are capable of participation in hydrolysis/condensation, such as metal or semi-metal alkoxide groups), followed by hydrolysis/condensation of the precursor groups.

In an example of the invention, an organic/ceramic hybrid composite is prepared by dissolving an organic polymer component in a suitable solvent and adding a ceramic sol precursor. The ratio of the organic polymer component to the ceramic sol precursor may be range from 95/5 to 5/95 on a weight basis, for instance from 80/20 to 20/80. Thickness of applied coating layers, as well as properties, can be controlled by varying the polymer to alkoxide concentration. By controlling such concentrations, layers can be applied in the micrometer or even nanometer range.

A solution of an acid or mixture of acids in water may be provided to accomplish hydrolysis and condensation of the ceramic sol precursor. Suitably, the pH of the acid(s) in solution is between about 3-6. The water is provided at a ratio of approximately one mole water per alkoxy equivalent in the ceramic sol source. The mixture may be stirred under reflux to form the sol, for instance for 4-24 hrs, after which it is used to prepare a coating, for instance by casting or coating onto a medical device substrate. The coating is thoroughly dried, optionally with addition of heat and/or vacuum to remove the solvent, and aged for several weeks to allow substantial completion of the ceramic condensation reaction. For a substrate such as a catheter shaft or balloon of a polymer such as Pebax® 6333, 7033 or 7233, the organic polymer may be, for instance, a Pebax® block copolymer such as the Pebax® grades 2533, 3533 or 4033, or a mixture thereof. For such a system the solvent may be an alcohol solvent such as butanol, propanol, or cyclohexanol or an amide solvent such as dimethylacetamide or a mixture of two or more such solvents. The ceramic sol precursor may be for instance tetraethoxysilane, zirconium isopropoxide, titanium isopropoxide or a mixture thereof. One example of a suitable acid is HCL at 0.05-0.3 moles per liter. The resulting coating has a good combination of toughness, adhesion to the substrate material and abrasion resistance.

The above discussion of acid catalysis is intended for illustrative purposes only. There are several different reaction mechanisms by which hydrolysis may be initiated, each leading to a different inorganic network architecture. For example, base catalysis may also be employed depending on the resultant chemical structure that is desired.

Multiple layers of the composite material disclosed herein can be deposited onto the shape form in order to produce a medical device having improved strength and to balance properties as desired such as strength and flexibility. They layers may be built by employing sequential coating or dipping steps.

Utilization of a functionalized polymer such as a maleic anhydride functional polymer can be advantageous to allow bonding between the sol-gel layer and a base layer. For example, creating layers having functionality which allows either hydrogen or covalent bonding between the layers can increase the adhesion between the layers. For example, functionalizing the soft segment of a polyether-block-amide copolymer with maleic anhydride or similar can be employed to enhance the interbonding between the sol-gel layer and the base layer. Other groups that may be grafted onto polymers include, but are not limited to, succinic anhydride, hexahydrophthalic anhydride, tetrahydrophthalic anhydride, dodecylsuccinic anhydride, phthalic anhydride, nadic anhydride, pyromellitic anhydride, etc. For grafted polymers for use in hydrogen or covalent bonding between layers, see commonly assigned U.S. Patent Publication No. 20070208155, the entire content of which is incorporated by reference herein.

For a variety of processing techniques, see, for example, commonly assigned U.S. Patent Publication No. 2007/0244501, the entire content of which is incorporated by reference herein in its entirety.

For the ceramic portion of the composite material, a network of metal or semi-metal oxides or mixed oxide compounds is typically formed. Any suitable network forming metal alkoxide may be employed in the formation of the sol-gel ceramic. The technique typically starts with a precursor material which may be an inorganic metallic salt or semi-metallic salt, a metallic or semi-metallic complex/chelate such as a metal acetylacetonate complex, a metallic or semi-metallic hydroxide, an organometallic and organo-semi-metallic compound such as a metal alkoxide, silicon alkoxides and acyloxide, etc. See U.S. Patent Publication No. 2007/0048348, incorporated by reference above. See C. J. Brinker; G. W. Scherer: “Sol-Gel Science”, Academic Press, San Diego 1990, for the basics of sol formation.

For example, an alkoxide of choice (such as a methoxide, ethoxide, isopropoxide, tert-butoxide, etc.) of a semi-metal or metal of choice (such as silicon, aluminum, zirconium, titanium, tin, hafnium, tantalum, molybdenum, tungsten, rhenium, iridium, etc.) may be dissolved in a suitable solvent, for example, in one or more alcohols, or other polar, protic or aprotic solvents, such as tetrahydrofuran, dioxane, dimethylformamide or butyl glycol, for example, may be employed. Suitable alcohols include, but are not limited to, ethanol, butanol, propanol, isopropanol, cyclohexanol, etc.

In some processes, fluorinated solvents, such as fluorinated hydrocarbons or fluorohydrocarbons can be employed.

Subsequently, a sol is formed, by hydrolysis/condensation reactions or by other condensation mechanisms. If desired, additional agents can be added, such as agents to control the viscosity and/or surface tension of the sol. For example, formamide or oxalic acid may reduce the surface tension. Such agents are known in the art. As a final stage the sol is converted to a gel by driving the condensation reaction further, for instance by drying the composition.

For a simplified reaction scheme see G. Kickelbick, “Concepts for the incorporation of inorganic building blocks into organic polymers on a nanoscale” Prog. Polym. Sci., 28 (2003) 83-114, the entire disclosure of which is incorporated herein by reference):

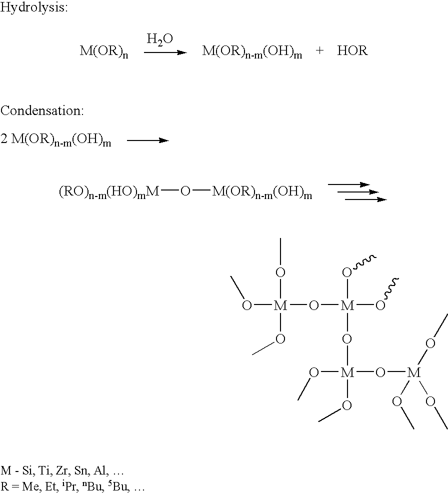

In general, R may be a hydrocarbon group, suitably an alkyl group of from 1-20 carbon atoms which optionally may be interrupted with one or more ether oxygen atoms, or an acyl group, for instance formyl, acetyl or benzoyl. Further, n is suitably equal to a valence of M and m is a positive number between 0 and n.

As noted above, any of a variety of metals can be employed in the formation of the ceramic portion, including, but not limited to, silicon, zirconium, titanium, aluminum, tin, hafnium, tantalum, molybdenum, tungsten, rhenium and/or iridium oxides, among others. In general, metal/semi-metal atoms (designated generally herein as M) within the ceramic phases are linked to one another via covalent linkages, such as M-O-M linkages, although other interactions are also commonly present including, for example, hydrogen bonding due to the presence of hydroxyl groups such as residual M-OH groups within the ceramic phases.

Different properties can be obtained depending on the type of metal or semi-metal oxides or alkoxides selected. For example, conductivity, radiopacity, ferromagneticity, etc. For example, metals such as vanadium (V), gold (Au), aluminum (Al), gallium (Ga), etc. can be employed to impart conductivity.

Gold, barium (Ba), bismuth (Bi), titanium (Ti), tantalum (Ta), tungsten (W), etc. can be employed to impart radiopacity.

Iron (Fe), cobalt (Co), nickel (Ni), gadolinium (Gd), dysprosium (Dy), etc. can be employed to impart ferromagneticity.

Specific materials can be selected so as to increase the mechanical strength. For example, ceramic materials including silicon (Si), titanium (Ti), aluminum (Al), zirconium (Zr), and so forth. One specific example of a organic-inorganic hybrid material including silicon is POSS (polyhedral oligomeric silsesquioxane).

These lists are intended for illustrative purposed only, and do not limit the scope of the present invention.

In some embodiments the organic-inorganic material is prepared by compounding a sol-gel ceramic precursor, optionally functionalized with an organic linking group, with a polymer component at elevated temperature, and subsequently processing the composition in a sol-gel technique to condense the ceramic network. If the ceramic precursor is functionalized with an organic linking group, such as isocyanate epoxy, a carbon-linked amino group or an ethylenically unsaturated group, a linking reaction to the organic polymer component may be run during the elevated temperature compounding step. Other functionalized sol-gel ceramic precursors that can form covalent linkages to the polymer during the elevated temperature compounding step may be alkoxysilanes having an ethylenically unsaturated group, for instance (meth)acryloxyethyltrimethoxysilane, (meth)acryloxypropyltriethoxysilane and 4-trimethoxysilylstyrene. Such functionalized sol-gel ceramic precursor can also be an alkoxysilane having a carbon-linked amino group, for instance 3-aminopropyltriethoxysilane. A SiH functionalized alkoxy silane such as triethoxysilane may be employed to form covalent linkages by hydrosilation.

In other alternatives the functionalized sol-gel ceramic precursor may have hydrolyzable groups other than alkoxide, for instance acyloxide.

In an alternative preparation the functionalized sol-gel ceramic precursor can be an active hydrogen reactive compound, for instance an isocyanate functional alkoxysilane, such as 3-isocyanatopropyltriethoxysilane or 2-isocyanotoethyltriethoxysilane. Epoxy functional ceramic precursors are also suitable, for example glycidoxypropyltrimethoxysilane. Such compounds maybe reacted with polymers that have active hydrogen groups, e.g. hydroxyl, thiol, primary amine, or secondary amine groups, to provide a covalent bond between the polymer and the functionalized alkoxide. The resulting polymer, now functionalized with alkoxysilane or other hydrolyzable silane groups, may then be incorporated into a ceramic network by hydrolysis/condensation of the alkoxysilane groups, suitably together with other sol-gel ceramic precursor compounds such as tetraethoxysilane, tetramethoxysilane, and/or monophenyltriethoxysilane, to produce the organic-inorganic hybrid material from which the inventive particulate materials are prepared. Further examples of preparation of such organic-inorganic hybrids are found in Honma, et al, Solid State Ionics, Vol 118, p 29-36, (1999); Honma, et al, Solid State Ionics, Vol 120, p. 255-264, (1999); Honma, et al, Journal of Membrane Science, Vol 185, p. 83-94, (2001); Huang, Wilkes, Polymer, Vol 30, p 2001-2012, (1989); Young, et al, Polymer, Vol 43, p 6101-6114, (2002); de Zea Bermudez, et al, Chem. Mater., Vol 11, p. 569-580, (1999); Yano, S., et al, Mater Sci Engng, Vol C6, p. 75-90, (1998); and Correia, et al, Solid State Ionics, Vol 156, p. 85-93, (2003).

Alternatively, the organic-inorganic hybrid can be made without covalent bonding therebetween, believed to be through weak hydrogen or Van der Waals bonding, by addition of polymer in an aqueous phase to a sol-gel process, for instance as described in Yano, S., et al, Mater Sci Engng, Vol C6, p. 75-90, (1998).

For the above ceramic precursors, see commonly assigned U.S. Patent Publication No. 2007/0072978, the entire content of which is incorporated by reference herein.

The sol-gel ceramic phase according to the invention can also be formed by hydrolysis of metal fluoroalkoxides such as alkaline earth metal fluoroalkoxides wherein at least some of the ceramic precursor materials have carbon-linked fluorocarbon groups to provide the medical device with a lower coefficient of friction while maintaining abrasion resistance and durability of the medical device surface, and without altering other bulk properties. For example, net-work forming metal compound wherein at least some of the molecules also have at least one fluorohydrocarbon group-metal atom link or bond which is not subject to hydrolysis, or at least subject only to partial hydrolysis may be employed. One example of a suitable fluorohydrocarbon group is a perfluorocarbyl segment.

These types of ceramic precursor materials are disclosed in commonly assigned U.S. Patent No. 2007/0048348, the entire content of which is incorporated by reference herein. Other examples of these types of ceramic precursor molecules having carbon-linked fluorocarbon groups are disclosed in U.S. Pat. Nos. 2,993,925; 3,491,134; 4,652,663; 5,250,322; 5,459,198; 5,876,686; and 6,713,186, each of which is incorporated by reference herein in its entirety.

Other descriptions of fluorinated silanes that may be employed are provided in Preparation of Super-Water-Repellent Fluorinated Inorganic-Organic Coating Films on Nylon 66 by the Sol-Gel Method Using Microphase Separation,” Satoh, K. et al., Journal of Sol-Gel Science and Technology, 27 (2003) 327-332; “Hybrid Organic-Inorganic Gels Containing Perfluoro-Alkyl Moieties,” Ameduri, Bruno, et al., Journal of Fluorine Chemistry, 104 (2000) 185-194; “Preparation and Surface Properties of Silica-Gel Coating Films Containing Branched-Polyfluoroalkylsilane,” Monde, Takashi, et al., Journal of Non-Crystalline Solids, 246 (1999) 54-64; “Hydrolysis and Condensation of Fluorine Containing Organosilicon, Kim,” Jae-Pil, et al., Optical Materials, 21 (2002) 445-450; “Synthesis and Surface Antimicrobial Activity of a Novel Perfluorooctylated Quaternary Ammonium Silane Coupling Agent,” Shao, Hui, et al., Journal of Fluorine Chemistry, 125 (2004) 721-724; “End-Capped Fluoroalkyl-Functional Silanes. Part II: Modification of Polymers and Possibility of Multifunctional Silanes,” Kawase, Tokuzo, et al., J. Adhesion Sci. Technol., Vol. 16, No. 8, pp. 1121-1140 (2002); “End-Capped Fluoroalkyl-Functional Silanes. Part I: Modification of Glass,” Kawase, Tokuzo, et al., J. Adhesion Sci. Technol., Vol. 16, No. 8, pp. 1103-1120 (2002) “A New Approach to Molecular Devices Using SAMs, LSMCD and Cat-CVD,” Nishikawa, T. et al., Science and Technology of Advanced Materials, 4 (2003) 81-89, all of which are incorporated by reference herein in their entirety.

Hydrolysis can occur without the addition of a catalyst. However, hydrolysis is most rapid and complete when a catalyst is added. Useful catalysts include, but are not limited to, mineral acids such as hydrochloric acid (HCl), carboxylic acids such as acetic acid and derivatives thereof such as trifluoroacetic acid, ammonia, potassium hydroxide (KOH), amines, hydrogen fluoride (HF), potassium fluoride (KF), etc. Furthermore, the rate and completion of the hydrolysis reaction is influenced to a greater degree by the strength and concentration of the acid or base employed.

If desired, additional agents can be added, such as agents to control the viscosity and/or surface tension of the sol. For example, formamide or oxalic acid may reduce the surface tension. Such agents are known in the art. As a final stage the sol is converted to a gel by driving the condensation reaction further, for instance by drying the composition.

Polymers for use in the composite regions of the present invention can have a variety of architectures, including cyclic, linear and branched architectures. Branched architectures include star-shaped architectures (e.g., architectures in which three or more chains emanate from a single branch point), comb architectures (e.g., architectures having a main chain and a plurality of side chains) and dendritic architectures (e.g., arborescent and hyperbranched polymers), among others. The polymers for use in the composite regions of the present invention can contain, for example, homopolymer chains, which contain multiple copies of a single constitutional unit, and/or copolymer chains, which contain multiple copies of at least two dissimilar constitutional units, which units may be present in any of a variety of distributions including random, statistical, gradient and periodic (e.g., alternating) distributions. Polymers containing two or more differing homopolymer or copolymer chains are referred to herein as “block copolymers.”

Many polymer materials that are commonly used in medical devices have hydroxyl, amide, carboxylic acid, ether, ester or other groups capable of forming hydrogen bonds with the ceramic network which can stabilize the system against macro-domain phase separation. For example, polyether-block-polyamides (e.g., PEBAX) and polyesters (e.g., polyethylene terephthalate) have terminal hydroxyl or carboxylic acid groups and internal amide groups. Ether and ester functionalities may also form hydrogen bonds with residual MOH groups in the ceramic phase. Moreover, as described in A. Lambert III, et al, “[Poly(ethylene terephthalate) ionomer]/Silicate Hybrid Materials via Polymer-in Situ Sol-Gel Reactions,” J. Applied Polymer Science, 84, 1749-1761 (2002), incorporated herein by reference in their entirety, ionic bonds in a polymer (for instance as provided in a polyolefin ionomer or a polyester ionomer) can also interact with the ceramic network to influence thermal, mechanical, electrical and/or chemical properties of the composite. In some embodiments, the polymer selected has polyether or polyamide segments.

Polymers for use in the composite regions of the present invention may be selected, for example, from one or more of the following: polycarboxylic acid polymers and copolymers including polyacrylic acids; acetal polymers and copolymers; acrylate and methacrylate polymers and copolymers (e.g., n-butyl methacrylate); cellulosic polymers and copolymers, including cellulose acetates, cellulose nitrates, cellulose propionates, cellulose acetate butyrates, cellophanes, rayons, rayon triacetates, and cellulose ethers such as carboxymethyl celluloses and hydroxyalkyl celluloses; polyoxymethylene polymers and copolymers; polyimide polymers and copolymers such as polyether block imides and polyether block amides, polyamidimides, polyesterimides, and polyetherimides; polysulfone polymers and copolymers including polyarylsulfones and polyethersulfones; polyamide polymers and copolymers including nylon 6,6, nylon 12, polycaprolactams and polyacrylamides; resins including alkyd resins, phenolic resins, urea resins, melamine resins, epoxy resins, allyl resins and epoxide resins; polycarbonates; polyacrylonitriles; polyvinylpyrrolidones (cross-linked and otherwise); polymers and copolymers of vinyl monomers including polyvinyl alcohols, polyvinyl halides such as polyvinyl chlorides, ethylene-vinyl acetate copolymers (EVA), polyvinylidene chlorides, polyvinyl ethers such as polyvinyl methyl ethers, polystyrenes, styrene-maleic anhydride copolymers, vinyl-aromatic-olefin copolymers, including styrene-butadiene copolymers, styrene-ethylene-butylene copolymers (e.g., a polystyrene-polyethylene/butylene-polystyrene (SEBS) copolymer, available as Kraton® G series polymers), styrene-isoprene copolymers (e.g., polystyrene-polyisoprene-polystyrene), acrylonitrile-styrene copolymers, acrylonitrile-butadiene-styrene copolymers, styrene-butadiene copolymers and styrene-isobutylene copolymers (e.g., polyisobutylene-polystyrene and polystyrene-polyisobutylene-polystyrene block copolymers such as those disclosed in U.S. Pat. No. 6,545,097 to Pinchuk), polyvinyl ketones, polyvinylcarbazoles, and polyvinyl esters such as polyvinyl acetates; polybenzimidazoles; ethylene-methacrylic acid copolymers and ethylene-acrylic acid copolymers, where some of the acid groups can be neutralized with either zinc or sodium ions (commonly known as ionomers); polyalkyl oxide polymers and copolymers including polyethylene oxides (PEO); polyesters including polyethylene terephthalates and aliphatic polyesters such as polymers and copolymers of lactide (which includes lactic acid as well as d-, l- and meso lactide), epsilon-caprolactone, glycolide (including glycolic acid), hydroxybutyrate, hydroxyvalerate, para-dioxanone, trimethylene carbonate (and its alkyl derivatives), 1,4-dioxepan-2-one, 1,5-dioxepan-2-one, and 6,6-dimethyl-1,4-dioxan-2-one (a copolymer of poly(lactic acid) and poly(caprolactone) is one specific example); polyether polymers and copolymers including polyarylethers such as polyphenylene ethers, polyether ketones, polyether ether ketones; polyphenylene sulfides; polyisocyanates; polyolefin polymers and copolymers, including polyalkylenes such as polypropylenes, polyethylenes (low and high density, low and high molecular weight), polybutylenes (such as polybut-1-ene and polyisobutylene), polyolefin elastomers (e.g., santoprene), ethylene propylene diene monomer (EPDM) rubbers, poly-4-methyl-pen-1-enes, ethylene-alpha-olefin copolymers, ethylene-methyl methacrylate copolymers and ethylene-vinyl acetate copolymers; fluorinated polymers and copolymers, including polytetrafluoroethylenes (PTFE), poly(tetrafluoroethylene-co-hexafluoropropene) (FEP), modified ethylene-tetrafluoroethylene copolymers (ETFE), and polyvinylidene fluorides (PVDF); silicone polymers and copolymers; thermoplastic polyurethanes (TPU); elastomers such as elastomeric polyurethanes and polyurethane copolymers (including block and random copolymers that are polyether based, polyester based, polycarbonate based, aliphatic based, aromatic based and mixtures thereof, examples of commercially available polyurethane copolymers include Bionate®, Carbothane®, Tecoflex®, Tecothane®, Tecophilic®, Tecoplast®, Pellethane®, Chronothane® and Chronoflex®); p-xylylene polymers; polyiminocarbonates; copoly(ether-esters) such as polyethylene oxide-polylactic acid copolymers; polyphosphazines; polyalkylene oxalates; polyoxaamides and polyoxaesters (including those containing amines and/or amido groups); polyorthoesters; biopolymers, such as polypeptides, proteins, polysaccharides and fatty acids (and esters thereof), including fibrin, fibrinogen, collagen, elastin, chitosan, gelatin, starch, glycosaminoglycans such as hyaluronic acid; as well as further copolymers of the above. In accordance with some embodiments of the invention the polymer is an organic polymer or an organic polymer modified with M(OR)ₓ groups where M and R are as defined subsequently herein.

The composite material according to the invention may also incorporate at least one therapeutic agent therein. This may be particularly advantageous wherein interpenetrating or semi-interpenetrating networks are formed between the polymer and the sol-gel derived ceramic. In this case, the therapeutic agent(s) can be entrapped therein and released upon exposure to a bodily fluid.

As used herein, the term “therapeutic agent” may be interchangeably used with a variety of terms including, but not limited to, “beneficial agents”, “drugs,” “bioactive agents”, “pharmaceutically active agents,” and other related terms as are known in the art.

These terms include genetic therapeutic agents, non-genetic therapeutic agents and cells.

Some specific therapeutic agents include anti-thrombotic agents, anti-proliferative agents, anti-inflammatory agents, anti-migratory agents, agents affecting extracellular matrix production and organization, antineoplastic agents, anti-mitotic agents, anesthetic agents, anti-coagulants, vascular cell growth promoters, vascular cell growth inhibitors, cholesterol-lowering agents, vasodilating agents, and agents that interfere with endogenous vasoactive mechanisms.

Specific examples of therapeutic agents include paclitaxel, sirolimus or rapamycin, everolimus, tacrolimus, Epo D, dexamethasone, estradiol, halofuginone, cilostazole, geldanamycin, ABT-578 (Abbott Laboratories), trapidil, liprostin, Actinomycin D, Resten-NG, Ap-17, abciximab, clopidogrel, Ridogrel, beta-blockers, bARKct inhibitors, phospholamban inhibitors, Serca 2 gene/protein, among others. Numerous additional therapeutic agents useful for the practice of the present invention are also disclosed in U.S. Patent Application 2004/0175406, commonly assigned U.S. Patent Application 2004/0215169 and U.S. Pat. No. 6,855,770, each of which is incorporated by reference herein in its entirety. A wide range of therapeutic agent loadings can be used in connection with the medical devices of the present invention, with the therapeutically effective amount being readily determined by those of ordinary skill in the art and ultimately depending, for example, upon the condition to be treated, the age, sex and condition of the patient, the nature of the therapeutic agent, the nature of the composite region(s), the nature of the medical device, and so forth.

Optionally, it is know to link a therapeutic agent(s) to the polymer itself See, for example, commonly assigned U.S. Patent Publication No. 20070178136, the entire content of which is incorporated by reference herein in its entirety.

The composite materials according to the invention may be employed in the formation of any catheter assembly or component thereof including expandable balloon members that are employed in PTCA and in the delivery of implantable medical devices such as stents.

Other medical devices which may be made in accordance with the invention include, but are not limited to, guide wires, filters (e.g., vena cava filters), stents (including coronary artery stents, peripheral vascular stents such as cerebral stents, urethral stents, ureteral stents, biliary stents, tracheal stents, gastrointestinal stents and esophageal stents), stent grafts, vascular grafts, vascular access ports, embolization devices including cerebral aneurysm filler coils (including Guglilmi detachable coils and metal coils), myocardial plugs, pacemaker leads, left ventricular assist hearts and pumps, total artificial hearts, heart valves, vascular valves, tissue bulking devices, sutures, suture anchors, anastomosis clips and rings, tissue staples and ligating clips at surgical sites, cannulae, metal wire ligatures, orthopedic prosthesis, joint prostheses, as well as various other medical devices that are adapted for implantation or insertion into the body.

The above disclosure is intended to be illustrative and not exhaustive. This description will suggest many variations and alternatives to one of ordinary skill in this art. Those familiar with the art may recognize other equivalents to the specific embodiments described herein which equivalents are also intended to be encompassed by the claims.

1. A process for forming at least a portion of a medical device comprising
a) providing an eliminatable a shape form, the shape form defining the shape of the at least a portion of the medical device;
b) depositing a composition comprising at least one polymer and at least one hydrolyzed and partially condensed sol-gel ceramic precursor onto said eliminatable shape form to form at least one first layer, the at least one first layer defining the shape of said at least a portion of the medical device;
c) condensing the hydrolyzed, partially condensed ceramic precursor to form a composite polymer-ceramic derived sol-gel; and
d) removing the shape-form material to provide said at least a portion of said medical device composed of the polymer-sol-gel derived ceramic composite.

2. The process of claim 1 wherein multiple layers of said composite are deposited onto said eliminatable shape form.

3. The process of claim 1 wherein said composite is deposited in a layer-by-layer construct.

4. The process of claim 1 wherein said ceramic precursor comprises at least one oxide network of silicon, aluminum, zirconium, hafnium, titanium, tin, gold, tantalum, molybdenum, tungsten, rhenium, iridium, vanadium, gallium, barium, bismuth, iron, cobalt, gadolinium, dysprosium, nickel, fluorine, and mixtures thereof.

5. The process of claim 1 wherein said ceramic precursor comprises at least one metal that is ferromagnetic.

6. The process of claim 1 wherein said ceramic precursor comprises at least one metal that is radiopaque.

7. The process of claim 1 wherein said ceramic precursor comprises at least one metal that is conductive.

8. The process of claim 1 wherein said polymer has a plurality of hydroxyl, amide, carboxylic acid, ester or ether groups thereon.

9. The process of claim 1 wherein said polymer comprises segments of polyamide or polyether of both.

10. The process of claim 1 wherein said polymer-sol-gel derived ceramic form an interpenetrating or semi-interpenetrating network.

11. The process of claim 1 wherein said polymer-sol-gel derived ceramic composite further comprises a therapeutic agent.

12. The process of claim 1 wherein said medical device is an expandable medical balloon, a stent, or a catheter.

13. A method of making at least a portion of a medical device from a composite material including at least one polymer material and at least one ceramic, the method including:
providing an eliminatable shape form;
depositing at least one layer of a composite material comprising at least one polymer material and at least one sol-gel derived ceramic on said surface of said eliminatable shape form; and
removing said eliminatable shape form;
wherein said at least a portion of said medical device is formed from said composite material.

14. The method of claim 13 comprising depositing a plurality of layers of said composite material on said surface of said eliminatable shape form.

15. The method of claim 13 wherein said medical device is an expandable balloon member, a stent or a catheter.

16. A medical device or a body portion thereof formed from a composite material, the composite material comprising at least one polymer and at least one sol-gel derived ceramic material.

17. The medical device of claim 16 wherein said device is an expandable balloon member, a stent or a catheter.

18. The medical device of claim 16 wherein said at least one polymer and at least one sol-gel derived ceramic form an interpenetrating of semi-interpenetrating network.

19. The medical device of claim 16 wherein said at least one composite material further comprises at least one therapeutic agent.

20. The medical device of claim 16 wherein said medical device or a portion thereof is formed of a plurality of layers of said composite material.

21. The medical device of claim 16 wherein said at least one sol-gel derived ceramic comprises at least one oxide network of silicon, aluminum, zirconium, hafnium, titanium, tin, gold, tantalum, molybdenum, tungsten, rhenium, iridium, vanadium, gallium, barium, bismuth, iron, cobalt, gadolinium, dysprosium, nickel, fluorine, and mixtures thereof.

22. The medical device of claim 16 wherein said sol-gel derived ceramic comprises at least one metal that is ferromagnetic.

23. The medical device of claim 16 wherein said sol-gel derived ceramic comprises at least one metal that is radiopaque.

24. The medical device of claim 16 wherein said sol-gel derived ceramic comprises at least one metal that is conductive.

25. The medical device of claim 16 wherein said polymer has a plurality of hydroxyl, amide, carboxylic acid, ester or ether groups thereon.

26. The medical device of claim 16 wherein said polymer comprises segments of polyamide or polyether of both.
